(19) United States
(12) Patent Application Publication
(10) Pub. No.: US 2005/0220753 A1
(43) Pub. Date: Oct. 6, 2005

(54) Hydrophilic polymers-flavoids conjugates and pharmaceutical compositions comprising them

(75) Inventors: Shishan Ji, Beijing (CN); Dequan Zhu, Beijing (CN)

Correspondence Address: MCKEE, VOORHEES & SEASE, P.L.C., 801 GRAND AVENUE, SUITE 3200, DES MOINES, IA, 50309-2721, US

(73) Assignee: BEIJING JIANKAI TECHNOLOGY CO., LTD, Beijing (CN)

(21) Appl. No.: 10/508,866

(22) PCT Filed: Mar. 20, 2003
(86) PCT No.: PCT/CN03/00206

§ 371 Date: May 11, 2005

(30) Foreign Application Priority Data
Mar. 22, 2002 (CN) PCT CN02 00186

(51) Int. Cl. A61K 31/765; C08G 63/48

(52) U.S. Cl. 424/78.3; 525/54.2

(57) ABSTRACT

The present invention discloses conjugates of polyethylene glycol and flavone or flavonoid drugs. The conjugates are formed by attaching PEG to the flavone derivatives such as puerarin, daidzein, baicalein and baicalin to improve their water solubility and circulation half-lives in the body. Because PEG is non-toxic and non-immunogenic, PEG conjugated drug molecules will have better therapeutic effects and convenience in administration.

### FIELDS OF THE INVENTION

The present invention relates to conjugates of hydrophilic polymers and flavone derivatives, especially the conjugates of hydrophilic polymers and small flavone or flavonoid molecules, such as puerarin, daidzein, scutellarein, scullcap flavone II, baicalein, baicalin and the like. The present invention also relates to pharmaceutical compositions comprising the conjugates.

### BACKGROUND OF THE INVENTION

Polyethylene glycol (PEG) derivatives have been widely used to conjugate proteins, peptides and other therapeutic agents to prolong their physiological half-lives and lower their immunogenicity and toxicity. Clinically, PEG and its derivatives have been widely used as carriers to formulate many commercial drugs. The methods of conjugating PEG to drug molecules have made great progress in the last 10 years and have been applied to many officially approved drugs. For example, PEG-intron®, a conjugate of PEG to α-interferon, exhibits a longer circulation half-life and a better therapeutic effect. A conjugate of PEG to paclitaxel reduces the toxicity and increases the bioactivity of the latter. The metabolism of PEG is known clearly and it is well known that PEG is a safe drug modifier.

A process called PEGylation is often applied when conjugating PEG to drug molecules. Namely, one or two of the terminal groups of the PEG are activated to form a proper functional group which is reactive to at least one functional group of the drug and can form a stable bond with it.

Flavones and flavonoids are widely used in the pharmaceutical field. Many of them have the same main structure: 2-phenyl-benzyl-benzo-γ-pyrone represented by the following formula:

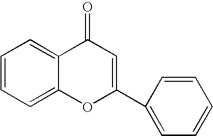

Yellow pigments of many plants are flavones and derivatives thereof containing multiple hydroxyl groups. Due to the phenyl substituent at different positions, some are classified as iso-flavones (3-phenyl-benzo-γ-pyrone) and other derivatives.

As extracted components of natural medicines, flavone derivatives, such as Puerarin, Baicalin and the like have been widely used to treat various diseases. They show distinct therapeutic effects on hypertension, angina, acute miocardial infarction and other cardiovascular diseases. But these flavone derivatives also have fast absorption and elimination rates. For example, in an intravenous injection test of rats, Baicalein's absorption half-life is 13 minutes and its elimination half-life is 42 minutes. The intravenously infused Puerarin has an elimination half-life of 74 minutes in human.

Therefore, it is really necessary to improve pharmacological half-lives of flavones and derivations thereof, to enhance their stability and probability of reaching target sites, to improve their water solubility, to change their route of administration, and to improve their bioavailability.

### SUMMARY OF THE INVENTION

In one aspect of the present invention, there is provided a conjugate of a hydrophilic polymer and a flavone drug, which is represented by the following formula:
(P-L [symbol] ₙD
wherein
P is a linear or branched hydrophilic polymer;
n is an integer from 1 to 10;
D is a flavone drug, preferably selected from the group consisting of puerarin, daidzein, scutellarein, scullcap flavone II, baicalein and baicalin; and
L is a linking group.

In another aspect of the invention, there is provided a conjugate of a hydrophilic polymer and puerarin represented by formula II: 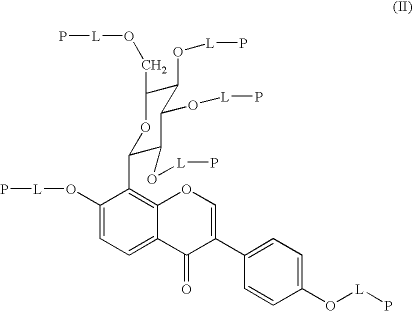
wherein
P is independently H or a hydrophilic polymer, with a proviso that all of the Ps are not H simultaneously; and
L is a linking group.

In still another aspect of the invention, there is provided a conjugate of a hydrophilic polymer and daidzein represented by formula III: 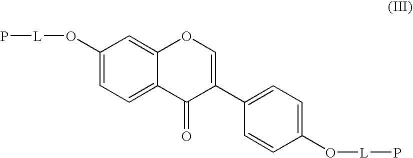
wherein
P is independently H or hydrophilic polymer, with a proviso that all of the Ps are not H simultaneously; and
L is a linking group.

In still another aspect of the invention, there is provided a conjugate of a hydrophilic polymer and baicalein represented by formula IV: 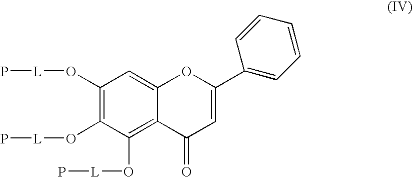
wherein
P is independently H or a hydrophilic polymer, with a proviso that all of the Ps are not H simultaneously; and
L is a linking group.

In still another aspect of the invention, there is provided a conjugate of a hydrophilic polymer and baicalin represented by formula V: 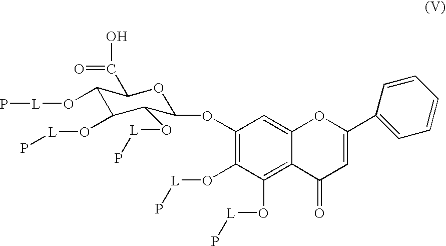
wherein
P is independently H or hydrophilic polymers, with a proviso that all of the Ps are not H simultaneously; and
L is a linking group.

In still another aspect of the invention, there is provided a conjugate of a hydrophilic polymer and baicalin represented by formula VI: 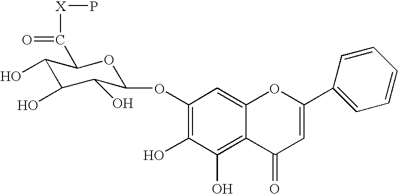
wherein
P is a linear or branched hydrophilic polymer; and
X is a linking moiety between the hydrophilic polymer and baicalin, such as NH or O.

In still another aspect of the invention, there are provided pharmaceutical compositions comprising the above conjugates as active ingredients.

The conjugates of the present invention prolong the therapeutic half-lives of the drugs in vivo through attachment to hydrophilic polymers. Also, the hydrophilic polymers can provide protection for the drugs conjugated thereto, improve the drugs' stability and hydrophilicity, prolong the drug's therapeutic life in vivo, and improve the drug's bioavailability in living bodies.

### BRIEF DESCRIPTION OF THE DRAWINGS

FIG. 1 shows the synthesis of actived polyethylene glycol derivatives.

FIG. 2 shows the synthesis of the conjugate of PEG and baicalein.

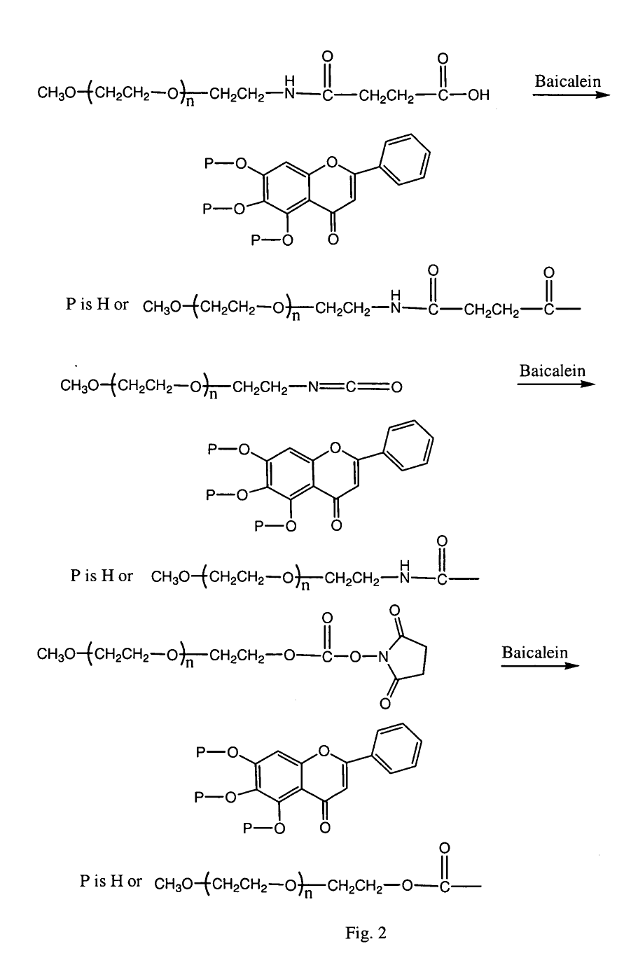

FIG. 3 shows the synthesis of the conjugate of PEG and baicalin.

### DETAILED DESCRIPTION OF THE INVENTION

In the conjugates of the present invention, the hydrophilic polymers can be any substantially non-antigenic polymer, including, for example, polyethylene glycol, polypropylene glycol, polyvinyl alcohol, polyacrylmorpholine or copolymers thereof, with polyethylene glycol being preferable.

The general structure of polyethylene glycol (PEG) is as shown in the formula below: 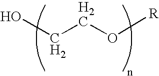
wherein
R is H, a C₁₋₁₂ alkyl or a cycloalkyl; and
n is an integer, representing the degree of polymerization;

As a lower alkyl, R can be any lower alkyl group having 1-6 carbon atoms, for example, methyl, ethyl, n-propyl, iso-propyl, n-butyl, iso-butyl, n-pentyl, or n-hexyl. As a cycloalkyl, R is preferably a cycloalkyl containing 3-7 carbon atoms, for example, cyclopropyl, cyclobutyl, and cyclohexyl. The preferred cycloalkyl is a cyclohexyl group. R the most preferred is a methyl group, with the formed compound being methoxy-polyethylene glycol (mPEG).

PEGs are usually measured by molecular weight. It is preferred that the molecular weight of PEG which forms the conjugates falls in the range from 300 to 60000 Daltons, which means n is about 6 to 1300. It is more preferred that n is 28, 112 and 450, respectively corresponding to molecular weights of 1325, 5000 and 20000 respectively. Because of the potential non-homogeneity of the starting PEGs, which are usually defined by their molecular weights rather than the self-repeating unit n, PEGs are normally characterized with a weight average molecular weight, rather than their self-repeating units represented by n. The starting PEG compounds with different molecular weights are readily synthesized using methods known in the art. They are also commercially available.

In addition to linear polymers, polymers having branched or other structures, such as Y-shaped branched and U-shaped branched PEGs, can be used in the modification of flavone drug structures. Suitable structures of PEGs are selected depending on the properties of the particular drug molecules.

In the present invention, the hydrophilic polymers generally have hydroxyl groups. So, the hydroxy groups of the hydrophilic polymers need to be activated to form terminal groups capable of reacting with hydroxy groups of the flavones. These functional groups will determine the conjugation framework and the use of the conjugates. For the conjugation, the following methods can be used to modify the terminal functional group. The following description is based on polyethylene glycol.

Amination

An amino group, which has greater reactivity, takes the place of hydroxyl group after amination. The amino group is especially important when the polymer reacts with a molecule having a carboxylic acid group to form a conjugate. 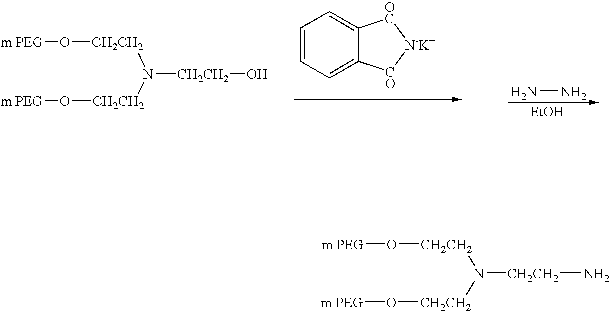
Carboxylation

After carboxylation, the carboxylic acid groups formed on the hydrophilic polymers help improving their reactivity, and make them capable of conjugating to molecules having amino or hydroxyl groups.

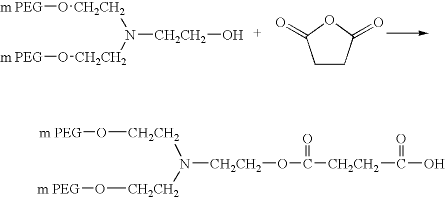

If amino acids are used as the starting materials, the activated polymers will have terminal carboxyl groups. Especially, if an acidic amino acid or a polymer containing an acidic amino acid is used, multiple active carboxyl groups will be provided. Such structures will improve the loading of small molecules from natural medicines, and will achieve a sustained release effect by biodegradation in vivo.

Other Methods

Modifications through acyl chloride, hydrazine, maleimide, pyridine disulfide and the like can also be appropriately adopted as well. Such modifications can be performed with any method well known in this field.

Flavones and flavonoids have multiple hydroxyl groups. For example, the sugar moiety of Puerarin comprises multiple hydroxyl groups. In addition, there are active hydroxyl groups at 7- and 4′-positions. These hydroxyl groups can be used as linking sites to polymers.

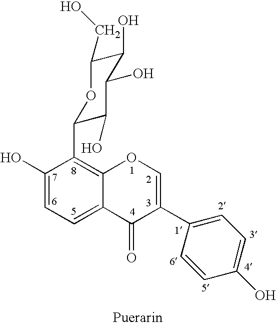

The other flavones, such as Daidzein, Scutellarein, Scullcap flavone II, Baicalein, Baicalin and the like, also have multiple hydroxyl groups, which can also be used as linked sites to polymers.

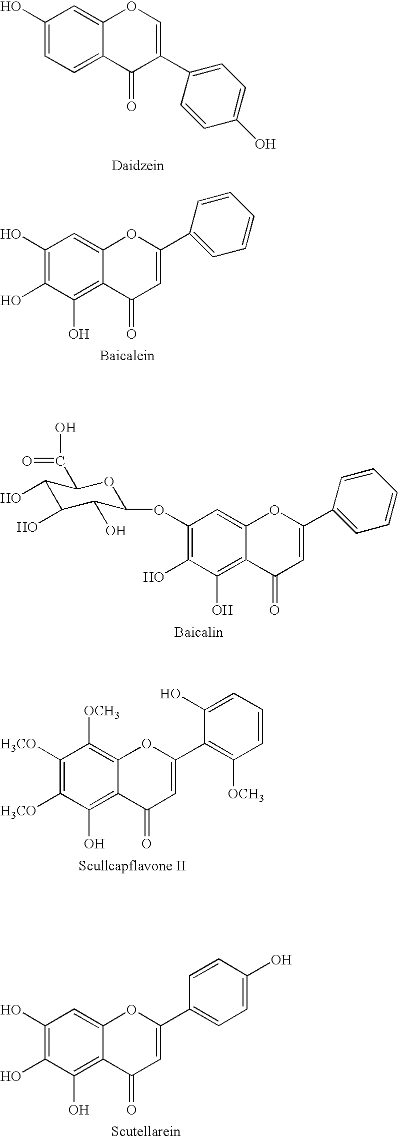

These hydroxy containing molecules can be conjugated to polymers through an ester group, a carbonate group, an amide group and the like, to protect the drug molecules and enable other useful applications. In the conjugates according to the present invention, the linking group L can be selected from the group consisting of ester, carbonate, ether, carboxylic acid, carbamate, and acetal groups, to link with hydroxy groups of the flavone drugs. According to the present invention, the term “flavone drugs” refers to flavones, flavonoids, iso-flavones, and any possible prodrugs thereof.

Hydrophilic polymers can be conjugated to drug molecules through an esterification reaction. This process can be illustrated as follows:

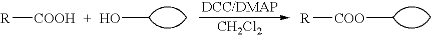

The ester group can be eliminated by biodegradation in vivo, and thereby the active ingredient is released.

According to a preferred embodiment of the present invention, the conjugates are provided that are formed by a hydrophilic polymer with puerarin, daidzein, baicalein or baicalin.

The conjugates of the present invention can be administered in the form of pure compounds or suitable pharmaceutical compositions including any dosage form for similar use via any acceptable route. According to another embodiment of the present invention, there are provided pharmaceutical compositions comprising the above conjugates.

The conjugates can be administered via oral, nasal, parenteral, topical, transdermal, rectal or injection routes in the form of solid, semisolid, lyophilized powder or liquid, for example, tablets, suppositories, pills, soft and hard gelatin capsules, powder, solution, suspension and aerosols. Preferably the unit dosage form is suitable for a precise-dosage and easy administration. The compositions may include conventional pharmaceutical carriers or excipients and one or more conjugates of the present invention as active ingredient(s). Furthermore, it also can include other agents, carriers and excipients.

Generally speaking, depending on the method of administration, the pharmaceutically acceptable compositions will include about 1-99 wt. % of the conjugate(s) of the present invention, and 99-1 wt. % of suitable pharmaceutical excipients. Preferably the composition include 5-75 wt. % of the conjugate(s) and the rest is any suitable pharmaceutical carrier or excipient.

The preferred method of administration is injection with a general daily dosage scheme, which can be adjusted depending on the severity of the disease to be treated. The conjugates of the present invention, or their pharmaceutically acceptable salts, may be formulated in the dosage form for injection by, for example, dissolving 0.5-50% of the active components in a liquid pharmaceutical carrier, such as water, saline, aqueous glucose, glycerol, ethanol and the like to form a solution or suspension.

If needed, the pharmaceutical compositions of the present invention can further include an adjuvant in a small amount, such as wetting agent, emulsifier, pH buffer, antioxidant and the like. For example, citric acid, sorbitan monolaurate, triethanolamine oleate, butylated hydroxytoluene and the like can be added.

The practical preparation methods of such dosage forms are known or obvious to those skilled in the art. For example, see Remington's Pharmaceutical Sciences, 18^{th} edition, (Mack Publishing Company, Easton, Pa, 1990). In any case, according to the techniques of the present invention, the composition applied will include an effective amount of the conjugate of the present invention for the treatment of a corresponding disease.

### EXAMPLES

The conjugates of the present invention and their preparation methods will be further described by referring to the following examples. However, these examples do not intend to limit the scope of the invention by any means. The scope of the present claimed invention can be determined from the claims.

### Example 1

### Synthesis of a Conjugate of Polyethylene Glycol and Puerarin through Ester Linkages

10 g of methoxypolyethylene glycol (mPEG, having a molecular weight of 5000) and 1 g of succinic anhydride were dissolved in 80 ml of dry acetonitrile, and then 0.5 ml of dry pyridine was added dropwise. The reaction mixture was stirred for 12 hours under the protection of nitrogen gas. Excess solvent was removed by rotary evaporation and the residue solid was added into 30 ml of isopropyl alcohol, the precipitated product was collected by filtering and dried under vacuum. Yield: 9.0 g (90%). NMR (DMSO): 3.5 (1H in PEG, br m), 3.24 (3H, s), 4.13(2H,t).

5 g of polyethylene glycol carboxylic acid (prepared in the former step, mPEG-COOH), 0.25 g of guerarin, 0.2 g of hydroxybenzotriazole, 0.2 g of 4-dimethylaminopyridine were dissolved in 50 ml of dry dichloromethane. 0.32 g of dicyclohexylcarbodiimide was added to the solution, and the mixture was stirred under the protection of nitrogen gas overnight. Excess solvent was removed by rotary evaporation and the residue solid was added into 20 ml of 1,4-dioxane, and the precipitate was removed by filtration. The filtrate was concentrated by rotary evaporation. 100 ml of isopropyl alcohol was added to the residue, and the precipitated product was filtered and dried under vacuum. Yield: 4.5g (90%). M.p: 60˜62° C.

### Example 2

### Synthesis of a Conjugate of Polyethylene Glycol and Daidzein through Carbonate Linkages

10 g of methoxypolyethylene glycol (having a molecular weight of 5000) and 0.25 g of N,N′-disuccinimidyl carbonate were dissolved in 100 ml of acetonitrile. 0.5 ml of dry pyridine was added to the solution dropwise. The reaction mixture was stirred under the protection of nitrogen gas overnight. The excess solvent was removed by rotary evaporation, and the residue was dried under vacuum. The dried solid residue was added to 30 ml of dry dichloromethane. The non-dissolved solid was removed by filtration and the remaining organic phase was washed once with sodium acetate buffer (0.1M, pH 5.5). The organic phase was dried over sodium sulfate, and concentrated. 20ml of ether was added into the residue. The product was collected by filtration and dried under vacuum. Yield: 9.0 g (90%). NMR (DMSO): 3.5 (br m, PEG), 3.24(s, 3H), 4.45 (t, 2H), 2.82 (s, 4H).

5 g of methoxypolyethylene glycol succinimidyl carbonate (MPEG-OCO-NHS) prepared in the former step and 0.125 g of daidzein were dissolved in 50 ml of dry acetonitrile. 0.2 g of 4-dimethylaminopyridine was added to the solution. The solution was heated and stirred under the protection of nitrogen gas overnight. The excess solvent was removed by rotary evaporation and 100 ml isopropyl alcohol was added to the residue. The product was collected by filtration and dried under vacuum. Yield: 4.5 g (90%). M.p: 57˜59 ° C.

### Example 3

### Synthesis of a Conjugate of Polyethylene Glycol and Scullcapflavone II through Carbamate Linkages

10 g of methoxypolyethylene glycol ethylamine (mPEG-NH₂, having a molecular weight of 5000) and 1 g of phosgene were dissolved in 80 ml of dry acetonitrile. 0.5 ml of dry pyridine was added to the solution. The reaction mixture was stirred under the protection of nitrogen gas overnight. The excess solvent was removed by rotary evaporation and the residue solid was added into 40 ml of ether. The precipitated product was collected by filtration and dried under vacuum. Yield: 9.5 g (95%). NMR (DMSO): 3.5 (br m, PEG), 3.24 (s, 3H), 3.18(t, 2H).

4.5 g of methoxypolyethylene glycol derivatives (MPEG-N═C═O) obtained from previous step, and 0.085g of scullcapflavone II were dissolved in 40 ml of dry acetonitrile. 0.5 ml of freshly distilled triethylamine (TEA) was added to the solution. The solution was stirred at room temperature under nitrogen atmosphere overnight. The excess solvent was removed by rotary evaporation and 100 ml isopropyl alcohol was added to the residue. The product was collected by filtration and dried under vacuum. Yield: 4.1 g (91%). M.p: 58˜60 ° C.

### Example 4

### Synthesis of a Conjugate of Methoxy-Polyethylene Glycol and Baicalein through Ester Linkages

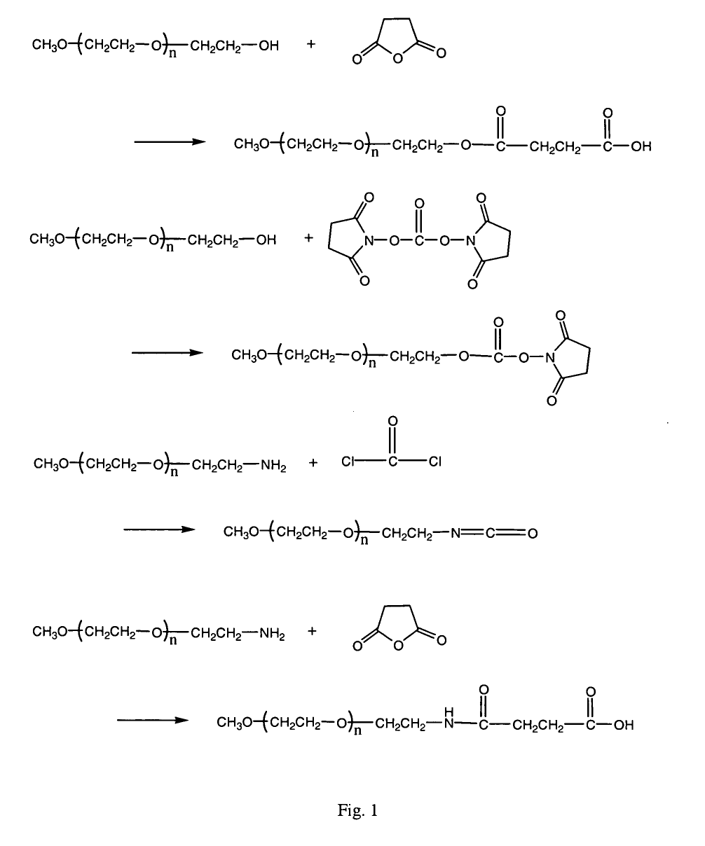

The synthesis is shown in FIG. 1. 10 g of methoxypolyethylene glycol ethylamine (mPEG-NH₂, having a molecular weight of 5000) and 1 g of succinic anhydride were dissolved in 80 ml of dry dichloromethane. 0.5 ml of dry pyridine was added to the solution. The reaction mixture was stirred 12 hours under the protection of nitrogen gas. The excess solvent was removed by rotary evaporation and the residue solid was added into 30 ml of isopropyl alcohol. The precipitated product was collected by filtration and dried under vacuum. Yield: 9.4 g (94%). NMR (DMSO): 3.5 (br m, PEG), 3.24 (s, 3H), 3.08(t, 2H).

4.5 g of methoxypolyethylene glycol carboxylic acid (obtain from former step), 0.085 g of baicalein, 0.2 g of hydroxybenzotriazole and 0.2 g of 4-dimethylaminopyridine were dissolved in 45 ml of dry methylene chloride, and then dicyclohexylcarbodiimide was added thereto. The solution was stirred at room temperature under the protection of nitrogen gas overnight. The excess solvent was removed by rotary evaporation and 20 ml of 1,4 dioxane was added to the residue. The precipitate was removed by filtration, and the filtrate was concentrated by rotary evaporation. 100 ml of isopropyl alcohol was added to the residue. The product was collected by filtration and dried under vacuum. Yield: 4.2 g (92%). Mp: 58-60° C.

### Example 5

### Synthesis of a Conjugate of Methoxy-Polyethylene Glycol and Baicalin

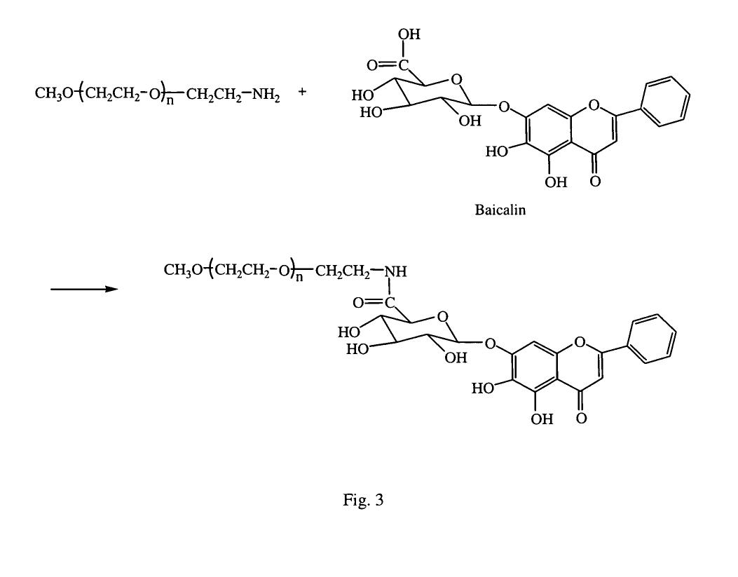

The synthesis is shown in FIG. 3. 5 g of methoxypolyethylene glycol ethylamine (mPEG-NH₂, having a molecular weight of 5000), 0.45 g of baicalein, 0.2 g of hydroxy-benzotriazole and 0.2 g of 4-dimethylaminopyridine were dissolved in 50 ml of dry methylene chloride, and then 0.25 g dicyclohexylcarbodiimide was added thereto. The solution was stirred at room temperature under the protection of nitrogen gas overnight. The excess solvent was removed by rotary evaporation and 20 ml of 1,4-dioxane was added to the residue. The precipitate was removed by filtration, and the filtrate was concentrated by rotary evaporation. 100 ml isopropyl alcohol was added to the residue. The product was collected by filtration and dried under vacuum. Yield: 4.6 g (92%). Mp: 59-62° C.

### Example 6

This example is to illustrate a preparation process of a typical parenteral composition. The composition comprises the conjugate of the present invention.

| Component | | |
|---|---|---|
| Conjugate prepared in Example 2 | 2 | g |
| 0.9% saline | to 100 | ml |

The conjugate prepared in Example 2 was dissolved in 0.9% saline to obtain a 100 ml solution for intravenous injection, which was filtered through a 0.2 μm membrane and packed aseptically.

1. A conjugate of a hydrophilic polymer and a flavone drug, which is represented by formula (I):
(P-L [symbol] ₙD (I)
wherein
P is a hydrophilic polymer;
n is an integer from 1 to 10;
D is a flavone drug; and
L is a linking group.

2. A conjugate of a hydrophilic polymer and puerarin represented by formula II: 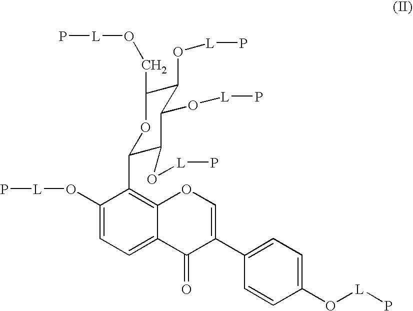
wherein
P is independently H or a hydrophilic polymer, with a proviso that all of the Ps are not H simultaneously; and
L is a linking group.

3. A conjugate of a hydrophilic polymer and daidzein represented by formula III: 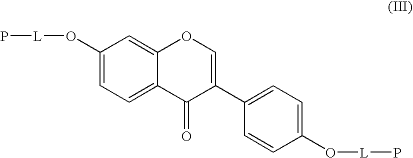
wherein
P is independently H or hydrophilic polymer, with a proviso that all of the Ps are not H simultaneously; and
L is a linking group.

4. A conjugate of a hydrophilic polymer and baicalein represented by formula IV: 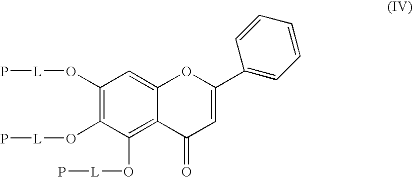
wherein
P is independently H or a hydrophilic polymer, with a proviso that all of the Ps are not H simultaneously; and
L is a linking group.

5. A conjugate of a hydrophilic polymer and baicalin represented by formula V: 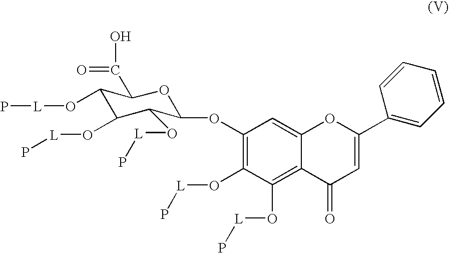
wherein
P is independently H or hydrophilic polymers, with a proviso that all of the Ps are not H simultaneously; and
L is a linking group.

6. A conjugate of a hydrophilic polymer and baicalin represented by formula VI: 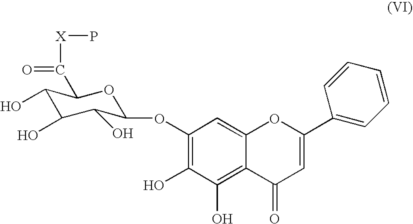
wherein
P is a linear or branched hydrophilic polymer; and
X is a linking moiety between the hydrophilic polymer and baicalin, such as NH or O.

7. The conjugate of claims 1 wherein the hydrophilic polymer is selected from the group consisting of polyethylene glycol, polypropylene glycol, polyvinyl alcohol, polyacryl morpholine and copolymers thereof.

8. The conjugate of claim 7 wherein the hydrophilic polymer is polyethylene glycol or a copolymer thereof.

9. The conjugate of claim 8, wherein polyethylene glycol has a molecular weight of from 300 to 60000.

10. The conjugate of claims 1, wherein the linking group L is selected from the group consisting of an ester, a carbonate, an ether, an amido, an amido ester, a carbamate and an acetal group.

11. A composition comprising the conjugate of claims 1 and a pharmaceutically acceptable carrier or excipient.

12. The composition of claim 11 further comprising other therapeutically active ingredients.

13. The composition of claim 11, wherein the composition is in a form of a solution, a tablet, a suspension or an aerosol.

14. The conjugate of claim 2 wherein the hydrophilic polymer is selected from the group consisting of polyethylene glycol, polypropylene glycol, polyvinyl alcohol, polyacryl morpholine and copolymers thereof.

15. The conjugate of claim 14 wherein the hydrophilic polymer is polyethylene glycol or a copolymer thereof.

16. The conjugate of claim 15, wherein polyethylene glycol has a molecular weight of from 300 to 60000.

17. The conjugate of claim 2, wherein the linking group L is selected from the group consisting of an ester, a carbonate, an ether, an amido, an amido ester, a carbamate and an acetal group.

18. The conjugate of claim 3 wherein the hydrophilic polymer is selected from the group consisting of polyethylene glycol, polypropylene glycol, polyvinyl alcohol, polyacryl morpholine and copolymers thereof.

19. The conjugate of claim 18 wherein the hydrophilic polymer is polyethylene glycol or a copolymer thereof.

20. The conjugate of claim 19, wherein polyethylene glycol has a molecular weight of from 300 to 60000.

21. The conjugate of claim 3, wherein the linking group L is selected from the group consisting of an ester, a carbonate, an ether, an amido, an amido ester, a carbamate and an acetal group.

22. The conjugate of claim 4 wherein the hydrophilic polymer is selected from the group consisting of polyethylene glycol, polypropylene glycol, polyvinyl alcohol, polyacryl morpholine and copolymers thereof.

23. The conjugate of claim 22 wherein the hydrophilic polymer is polyethylene glycol or a copolymer thereof.

24. The conjugate of claim 23, wherein polyethylene glycol has a molecular weight of from 300 to 60000.

25. The conjugate of claim 4, wherein the linking group L is selected from the group consisting of an ester, a carbonate, an ether, an amido, an amido ester, a carbamate and an acetal group.

26. The conjugate of claim 5 wherein the hydrophilic polymer is selected from the group consisting of polyethylene glycol, polypropylene glycol, polyvinyl alcohol, polyacryl morpholine and copolymers thereof.

27. The conjugate of claim 26 wherein the hydrophilic polymer is polyethylene glycol or a copolymer thereof.

28. The conjugate of claim 27, wherein polyethylene glycol has a molecular weight of from 300 to 60000.

29. The conjugate of claim 5, wherein the linking group L is selected from the group consisting of an ester, a carbonate, an ether, an amido, an amido ester, a carbamate and an acetal group.

30. The conjugate of claim 6 wherein the hydrophilic polymer is selected from the group consisting of polyethylene glycol, polypropylene glycol, polyvinyl alcohol, polyacryl morpholine and copolymers thereof.

31. The conjugate of claim 30 wherein the hydrophilic polymer is polyethylene glycol or a copolymer thereof.

32. The conjugate of claim 31, wherein polyethylene glycol has a molecular weight of from 300 to 60000.

33. The composition of claim 11 wherein the hydrophilic polymer is selected from the group consisting of polyethylene glycol, polypropylene glycol, polyvinyl alcohol, polyacryl morpholine and copolymers thereof.

34. The composition of claim 33 wherein the hydrophilic polymer is polyethylene glycol or a copolymer thereof.

35. The composition of claim 34, wherein polyethylene glycol has a molecular weight of from 300 to 60000.

36. The composition of claim 11, wherein the linking group L is selected from the group consisting of an ester, a carbonate, an ether, an amido, an amido ester, a carbamate and an acetal group.

37. A composition comprising the conjugate of claim 2 and a pharmaceutically acceptable carrier or excipient.

38. The composition of claim 37 wherein the hydrophilic polymer is selected from the group consisting of polyethylene glycol, polypropylene glycol, polyvinyl alcohol, polyacryl morpholine and copolymers thereof.

39. The composition of claim 38 wherein the hydrophilic polymer is polyethylene glycol or a copolymer thereof.

40. The composition of claim 39, wherein polyethylene glycol has a molecular weight of from 300 to 60000.

41. The composition of claim 37, wherein the linking group L is selected from the group consisting of an ester, a carbonate, an ether, an amido, an amido ester, a carbamate and an acetal group.

42. A composition comprising the conjugate of claim 3 and a pharmaceutically acceptable carrier or excipient.

43. The composition of claim 42 wherein the hydrophilic polymer is selected from the group consisting of polyethylene glycol, polypropylene glycol, polyvinyl alcohol, polyacryl morpholine and copolymers thereof.

44. The composition of claim 43 wherein the hydrophilic polymer is polyethylene glycol or a copolymer thereof.

45. The composition of claim 44, wherein polyethylene glycol has a molecular weight of from 300 to 60000.

46. The composition of claim 42, wherein the linking group L is selected from the group consisting of an ester, a carbonate, an ether, an amido, an amido ester, a carbamate and an acetal group.

47. A composition comprising the conjugate of claim 4 and a pharmaceutically acceptable carrier or excipient.

48. The composition of claim 47 wherein the hydrophilic polymer is selected from the group consisting of polyethylene glycol, polypropylene glycol, polyvinyl alcohol, polyacryl morpholine and copolymers thereof.

49. The composition of claim 48 wherein the hydrophilic polymer is polyethylene glycol or a copolymer thereof.

50. The composition of claim 49, wherein polyethylene glycol has a molecular weight of from 300 to 60000.

51. The composition of claim 47, wherein the linking group L is selected from the group consisting of an ester, a carbonate, an ether, an amido, an amido ester, a carbamate and an acetal group.

52. A composition comprising the conjugate of claim 5 and a pharmaceutically acceptable carrier or excipient.

53. The composition of claim 52 wherein the hydrophilic polymer is selected from the group consisting of polyethylene glycol, polypropylene glycol, polyvinyl alcohol, polyacryl morpholine and copolymers thereof.

54. The composition of claim 53 wherein the hydrophilic polymer is polyethylene glycol or a copolymer thereof.

55. The composition of claim 54, wherein polyethylene glycol has a molecular weight of from 300 to 60000.

56. The composition of claim 52, wherein the linking group L is selected from the group consisting of an ester, a carbonate, an ether, an amido, an amido ester, a carbamate and an acetal group.

57. A composition comprising the conjugate of claim 6 and a pharmaceutically acceptable carrier or excipient.

58. The composition of claim 57 wherein the hydrophilic polymer is selected from the group consisting of polyethylene glycol, polypropylene glycol, polyvinyl alcohol, polyacryl morpholine and copolymers thereof.

59. The composition of claim 58 wherein the hydrophilic polymer is polyethylene glycol or a copolymer thereof.

60. The composition of claim 59, wherein polyethylene glycol has a molecular weight of from 300 to 60000.
